(19) United States
(12) Patent Application Publication
(10) Pub. No.: US 2014/0277202 A1
(43) Pub. Date: Sep. 18, 2014

(54) Connecting Structure of a Spinal Detector for a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/845,252

(22) Filed: Mar. 18, 2013

(51) Int. Cl. A61B 17/70

(52) U.S. Cl. CPC A61B 17/7083
USPC 606/86 A

(57) ABSTRACT

A connecting structure of a spinal detector for a minimally invasive surgery contains: a positioning disc, a coupling member, and a plurality of metal bars. The positioning disc includes a plurality of holes defined on a front portion thereof in a sector arrangement. The coupling member is fixed on a rear portion of the positioning disc and includes a square seat and a holder. The square seat is mounted on the positioning disc and has the holder disposed thereon, and the holder has a circular boss formed thereon. The plurality of metal bars is circular and inserted in the plurality of holes of the positioning disc, and each metal bar includes a specific length.

### BACKGROUND OF THE INVENTION

1. Field of the Invention

The present invention relates to a connecting structure of a spinal detector for a minimally invasive surgery by which a positioning disc is attached on a skin, and then a plurality of metal bars are inserted into the skin via a plurality of holes of the positioning disc so as to fix the positioning disc, thus decreasing a wound area effectively.

2. Description of the Prior Art

A conventional connecting structure of a spinal detector is fixed on a wound resulting from a surgery, and the spinal detector is mounted on the conventional connecting structure, thus positioning the connecting structure.

As shown in FIG. 4, a conventional connecting structure 10 is a clamping structure 101, wherein the conventional connecting structure 10 includes an adjusting member 102 located at an upper end of a central position thereof, and a connecting member 103 is arranged on the adjusting member 102 so as to couple with a spinal detector 20. In operation, the clamping structure 101 is fixed a wound 30 resulting from a surgery, and then the connecting member 103 is in connection with the spinal detector 20 so as to detect a spine.

Nevertheless, the connecting structure is fixed on a large wound instead of a small wound. So, for the small wound requirement, an extra connecting structure has to be used on the small wound.

The present invention has arisen to mitigate and/or obviate the afore-described disadvantages.

### SUMMARY OF THE INVENTION

The primary object of the present invention is to provide a connecting structure of a spinal detector for a minimally invasive surgery which is capable of overcoming the shortcomings of the connecting structure of the spinal detector.

Another object of the present invention is to provide a connecting structure of a spinal detector for a minimally invasive surgery by which a positioning disc is attached on a skin, and then a plurality of metal bars are inserted into the skin via a plurality of holes of the positioning disc so as to fix the positioning disc, thus decreasing a wound area effectively.

To obtain the above objectives, a connecting structure of a spinal detector for a minimally invasive surgery contains a positioning disc, a coupling member, and a plurality of metal bars.

The positioning disc includes a plurality of holes defined on a front portion thereof in a sector arrangement.

The coupling member is fixed on a rear portion of the positioning disc and includes a square seat and a holder. The square seat is mounted on the positioning disc and has the holder disposed thereon, and the holder has a circular boss formed thereon.

The plurality of metal bars are circular and inserted in the plurality of holes of the positioning disc, and each metal bar includes a specific length.

### BRIEF DESCRIPTION OF THE DRAWINGS

FIG. 1 is a perspective view of the assembly showing a connecting structure of a spinal detector for a minimally invasive surgery according to a preferred embodiment of the present invention.

FIG. 2 is a cross sectional view showing the operation of the connecting structure of the spinal detector for the minimally invasive surgery according to the preferred embodiment of the present invention.

FIG. 3 is a perspective view showing the operation of the connecting structure of the spinal detector for the minimally invasive surgery according to the preferred embodiment of the present invention.

FIG. 4 is a perspective view of a conventional connecting structure of a spinal detector for a minimally invasive surgery.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

With reference to FIGS. 1-2, a connecting structure of a spinal detector for a minimally invasive surgery according to a preferred embodiment of the present invention comprises: a positioning disc 1, a coupling member 2, and a plurality of metal bars 3.

The positioning disc 1 includes a plurality of holes 11 defined on a front portion thereof in a sector arrangement.

The coupling member 2 is fixed on a rear portion of the positioning disc 1 and includes a square seat 21 and a holder 22. The square seat 21 is mounted on the positioning disc 1 and has the holder 22 disposed thereon, and the holder 22 has a circular boss 23 formed thereon.

The plurality of metal bars 3 are circular and inserted in the plurality of holes 11 of the positioning disc 1, and each metal bar 3 includes a specific length.

As shown in FIG. 3, in operation, the positioning disc 1 is attached on a skin 5, and then the plurality of metal bars 3 are inserted into the skin 5 via the plurality of holes 11 of the positioning disc 1 so as to fix the positioning disc 1. Thereafter, the coupling member 2, which is fixed on the rear portion of the positioning disc 1, is connected with a spinal detector 4 so as to clearly learn a position of the spine in a surgery.

While we have shown and described various embodiments in accordance with the present invention, it is clear to those skilled in the art that further embodiments may be made without departing from the scope of the present invention.

What is claimed is:

1. A connecting structure of a spinal detector for a minimally invasive surgery comprising:
a positioning disc including a plurality of holes defined on a front portion thereof in a sector arrangement;
a coupling member including a square seat and a holder; the square seat being mounted on the positioning disc, and the holder having a circular boss formed thereon;
a plurality of metal bars being circular and inserted in the plurality of holes of the positioning disc, and each metal bar including a length.
